(19) United States
(12) Patent Application Publication
(10) Pub. No.: US 2004/0259925 A1
(43) Pub. Date: Dec. 23, 2004

(54) Pharmaceutical compositions and methds for the prevention or treatment of cardiovascular, cardiopulmonary, pulmonary or renal diseases

(75) Inventors: Axel Riedel, Maselheim (DE); Josep-Maria Sendra, Wiesbaden (DE); Josef M.E. Leiter, Holzkirchen (DE); Stefan Kauschke, Biberach (DE); Michael Mark, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/757,295

(22) Filed: Jan. 14, 2004

Related U.S. Application Data
(60) Provisional application No. 60/446,695, filed on Feb. 11, 2003.
(60) Provisional application No. 60/503,317, filed on Sep. 16, 2003.

(30) Foreign Application Priority Data
Jan. 16, 2003 (DE) 10301371.7
Jul. 31, 2003 (DE) 10335027.6

(51) Int. Cl. A61K 31/4178; A61K 31/401

(52) U.S. Cl. 514/381; 514/423

(57) ABSTRACT

The present invention relates to a process for the prevention or treatment of cardiovascular, cardiopulmonary, pulmonary or renal diseases by improving endothelial function and achieving protection of organs, tissues and blood vessels in indications in which control of blood pressure and lipid levels are necessary, particularly in people in whom type 2 diabetes mellitus has been diagnosed or who are suspected of prediabetes, for preventing diabetes and prediabetes, or for the treatment of Metabolic Syndrome and insulin resistance in patients with normal blood pressure. The process comprises generally administering effective amounts of telmisartan or a polymorph or salt thereof and atorvastatin. The invention further relates to suitable pharmaceutical compositions which contain telmisartan or a polymorph or salt thereof and atorvastatin, as a combined preparation for simultaneous, separate or sequential use in the prevention or treatment of these diseases.

### RELATED APPLICATIONS

Benefit of DE 10301371.7, filed Jan. 16, 2003; DE 10335027.6, filed Jul. 31, 2003; U.S. Provisional Application No. 60/446,695, filed Feb. 11, 2003; and U.S. Provisional Application No. 60/503,317, filed Sep. 16, 2003, are hereby claimed.

### BACKGROUND OF THE INVENTION

The invention relates to: a process for the prevention or treatment of cardiovascular, cardiopulmonary, pulmonary or renal diseases, particularly in people in whom diabetes has been diagnosed or who are suspected of prediabetes, for preventing diabetes and prediabetes, or for the treatment of Metabolic Syndrome and insulin resistance in patients with normal blood pressure. The process comprises generally administering effective amounts of the angiotensin II receptor antagonist telmisartan and the HMG-CoA-reductase inhibitor atorvastatin or polymorphs or salts thereof to a person in need of treatment. The invention further relates to suitable pharmaceutical compositions which contain telmisartan and atorvastatin or polymorphs or salts thereof, as a combined preparation for simultaneous, separate or sequential use in the prevention or treatment of these diseases, as well as the combined use of telmisartan and atorvastatin or polymorphs or salts thereof for preparing a pharmaceutical composition for the prevention or treatment of these diseases.

Angiotensin II (ANG II) plays an important part in pathophysiology, particularly as the most potent agent for increasing blood pressure in humans. It is known that in addition to its effect of raising blood pressure ANG II also has growth-promoting effects which contribute to left ventricular hypertrophy, vascular thickening, atherosclerosis, kidney failure and stroke. On the other hand, bradykinin has vasodilating and tissue-protecting effects. Therefore, ANG II antagonists are suitable for the treatment of raised blood pressure and congestive heart failure in mammals. Examples of ANG II antagonists are described in EP-A-0 502 314, EP-A-0 253 310, EP-A-0 323 841, EP-A-0 324 377, U.S. Pat. No. 4,355,040 and U.S. Pat. No. 4,880,804. Examples of ANG II antagonists are candesartan, eprosartan, irbesartan, losartan, olmesartan, tasosartan, valsartan or telmisartan.

The antihypertensive and kidney-protecting effects of ANG II antagonists are described for example in the following publications:

W. Wienen et al.: Antihypertensive and renoprotective effects of telmisartan after long term treatment in hypertensive diabetic (D) rats, 2nd Int. Symposium on Angiotensin II Antagonism, Feb. 15-18, 1999, The Queen Elizabeth II Conference Center, London, UK, Book of Abstracts, Abstract No. 50;

J. Wagner et al.: Effects of AT1 receptor blockade on blood pressure and the renin angiotensin system in spontaneously hypertensive rats of the stroke prone strain, Clin. Exp. Hypertens., vol. 20 (1998), p. 205-221; and

M. Böhm et al.: Angiotensin II receptor blockade in TGR(mREN2)27: Effects of renin-angiotensin-system gene expression and cardiovascular functions, J. Hypertens., vol. 13 (8) (1995), p. 891-899.

Other renoprotective effects of ANG II antagonists which were found in first clinical trials are described in the following publications, for example:

S. Andersen et al.: Renoprotective effects of angiotensin II receptor blockade in type 1 diabetic patients with diabetic nephropathy, Kidney Int., vol. 57 (2) (2000), p. 601-606;

L. M. Ruilope: Renoprotection and renin-angiotensin system blockade in diabetes mellitus, Am. J. Hypertens., vol.10(12 PT 2) Suppl. (1997), p. 325-331; and

J. F. E. Mann: Valsartan and the kidney: Present and future, J. Cardiovasc. Pharmacol., vol. 33, Suppl. 1 (1999), p. 37-40.

Moreover the effects of ANG II antagonists on endothelial dysfunction are described in the following publications, for example:

E. L. Schiffrin et al.: Correction of arterial structure and endothelial dysfunction in human essential hypertension by the angiotensin receptor antagonist losartan, Circulation, vol. 101 (14) (2000), p. 1653-1659;

R. M. Touyz et al.: Angiotensin II stimulates DNA and protein synthesis in vascular smooth muscle cells from human arteries: role of extracellular signal-regulated kinases, J. Hypertens., vol.17(7) (1999), p. 907-916;

E. L. Schiffrin: Vascular remodelling and endothelial function in hypertensive patients: Effects of antihypertensive therapy, Scand. Cardiovasc. J., vol. 32, Suppl. 47 (1998) p. 15-21; and

Prasad: Acute and chronic angiotensin-1 receptor antagonism reverses endothelial dysfunction in atherosclerosis, Circulation, vol. 101 (2000), p. 2349 ff.

It is also known that ANG II antagonists selectively block the AT1 receptor, while the AT2 receptor which plays a part in anti-growth effects and tissue regeneration effects remains unaffected.

EP-A-1 013 273 also describes the use of AT1-receptor antagonists or AT2-receptor modulators for the treatment of diseases associated with an increase in the AT1-receptors in the sub-epithelial region or an increase in the AT2-receptors in the epithelium, particularly for the treatment of various lung diseases.

In another aspect it was found that hypertension is often present at the same time as hyperlipidaemia. Both symptoms are regarded as serious risk factors in the development of cardiovascular diseases, which often lead to unfavourable cardiovascular events.

High blood cholesterol levels and high blood lipid levels are involved for example in the start of atherosclerosis, a condition characterised by unevenly distributed lipid deposits inside the arteries, including the coronary, carotid and peripheral arteries.

This irregular lipid distribution is thus characteristic of coronary heart damage, cardiovascular diseases, the gravity and prevalence of which are also affected by the existence of diabetes, the sex of the person, cigarette smoking and left ventricular hypertrophy occurring as a side effect of hypertension (Wilson et al., Am. J. Cardiol., vol. 59(14) (1987), p. 91G-94G).

Type 2 diabetes mellitus is the manifestation of two pathophysiological phenomena, namely a reduced secretion of insulin from the beta cells of the pancreas and insulin resistance in the target organs of the liver, skeletal musculature and fatty tissue. As a rule there is a complex disruption of both components. The disease is diagnosed as fasting hyperglycaemia, i.e. the blood sugar concentration after 10-12 hours' fasting is above the threshold of 125 mg of glucose per dl of plasma. Controlled treatment of manifest type 2 diabetes can be achieved using compounds of the category of the thiazolidinediones (glitazones). These compounds improve the utilisation of circulating insulin and thus result in a lowering of the blood sugar levels (insulin sensitisers). At the same time the increased insulin levels are reduced by feedback mechanisms and in this way the load on the pancreas is relieved. Insulin sensitisers such as troglitazone, rosiglitazone or pioglitazone develop this activity by binding to specific nuclear receptors known as PPAR-gamma (Peroxisomal Proliferator Activated Receptor).

WO 95/06410 discloses the use of angiotensin II receptor antagonists for treating chronic inflammatory diseases including systemic autoimmune diseases. Diabetes is mentioned as one of a number of examples of systemic autoimmune diseases. The autoimmune diseases include type 1 diabetes mellitus which occurs mainly in young people under 30 years of age with a genetic predisposition, in whom insulinitis occurs under the influence of various factors with subsequent destruction of the B cells so that the pancreas can only produce a little insulin or none at all. Type 2 diabetes mellitus is not regarded as an autoimmune disease.

Because every other type 2 diabetes patient shows signs of coronary heart disease at the time of diagnosis, for example, the causes of diabetes are increasingly suspected to reside in a complex metabolic disorder which may be indicated by a number of risk factors such as abnormal glucose tolerance, increased fasting blood sugar, insulin resistance, high blood pressure, dyslipidaemia or centripetal obesity. The prevalence of insulin resistance is particularly marked in patients with hypertriglyceridaemia and low HDL (high-density lipoprotein)-cholesterol. Reference is made to pre-type 2 diabetes, metabolic syndrome, syndrome X or insulin resistance syndrome. In a first phase a reduced insulin response by the target organs causes an increase in the pancreatic insulin secretion in order to keep the blood sugar level in the normal range. After a number of years of excessive or increasing insulin production there comes a time when the insulin secretion by the beta cells of the pancreas cannot be increased any further. The phase of abnormal glucose tolerance then begins. The body can no longer absorb glucose peak values fast enough. Finally, if the fasting blood sugar remains persistently high, diabetes is manifest.

Angina pectoris, a condition characterised by severe constricting pains in the chest, often radiating out from the heart area to the left shoulder and down to the left arm, is frequently treated with a combination therapy of β-blockers, nitrate or calcium channel blockers together with a lipid lowering agent. Angina pectoris is often the result of cardiac ischaemia and is normally caused by coronary disease. When treated surgically, angina patients often suffer complications such as restenosis which is experienced either as a short term proliferative reaction to the trauma caused by the angioplasty or as a long-term progression of the arteriosclerotic process both in transplanted vessels and in angioplasty segments. Some possible treatments for lowering lipids and cholesterol are based on inhibiting the activity of the enzyme 3-hydroxy-3-methylglutaryl-coenzyme A-reductase (HMG-CoA-reductase), which catalyses the conversion of 3-hydroxy-3-methylglutaryl-coenzyme A into mevalonate, an early stage in the biosynthetic cholesterol metabolic pathway. Known inhibitors of HMG-CoA-reductase are for example compounds derived from a fungal metabolite the names of which end in “statin”, such as pravastatin, lovastatin, fluvastatin, simvastatin or atorvastatin.

Atorvastatin is a potent inhibitor of the enzyme 3-hydroxy-3-methylglutaryl-coenzyme A-reductase (HMG-CoA-reductase) and is known as a high-grade liver-selective inhibitor of cholesterol biosynthesis, the effect of which involves lowering Low Density Lipoprotein Cholesterol (LDL-C). These activities are the reason for the attractiveness of this molecule in the treatment of combined hyperlipidaemia, a normal atherogenic disorder in clinical practice, and thus also in preventing the progression of atheroma.

Investigations have also shown that lowering the LDL-C level provides protection against coronary heart diseases (cf. for example “Scandinavian Simvastatin Survival Study” or 4S study, published in The Lancet, vol. 344 (1994), p. 1383-1389, or the study “Prevention of coronary heart disease with privastatin in men with hypercholesterolemia”, published by Shepherd et al., in The New England Journal of Medicine, vol. 333 (1995), p. 1301-1307).

Other studies are being carried out to determine the protective effect of statins against the occurrence of heart attacks, strokes and coronary heart diseases in non-insulin-dependent diabetics; “Collaborative Atorvastatin Diabetes Study” or the CARDS study “Atorvastatin Versus Revascularisation Treatment”, the AVERT study and the “Anglo-Scandinavian Cardiac Outcomes trial” or ASCOT study.

Since high blood pressure often occurs together with hyperlipidaemia or signs of type 2 diabetes, as already mentioned, and since these signs are main risk factors for the development of cardiovascular diseases which often lead to unfavourable cardiovascular events, it would be beneficial for the patient to have access to a single therapy which prevents or treats these conditions. It would also be advantageous if the combination therapy also brought about an improvement in the prevention or treatment of cardiopulmonary, pulmonary or renal diseases for which ANG II antagonists have been found to be effective.

The aim of the present invention is to provide pharmaceutical compositions which are suitable both for the treatment of high blood pressure and also for the treatment of hyperlipidaemia and simultaneously for the treatment of manifest type 2 diabetes and also for the treatment of first indications of the complex metabolic disorder of prediabetes and hence may be used to prevent type 2 diabetes mellitus.

Combined treatments and corresponding compositions which contain HMG-CoA-reductase inhibitors and ANG II antagonists have already been proposed.

WO-95/26188 describes a method of treating atherosclerosis and reducing cholesterol, using an HMG-CoA-reductase-inhibitor and an ANG II antagonist. Pravastatin, simvastatin and lovastatin are mentioned as possible HMG-CoA-reductase inhibitors which may be used. Losartan is mentioned as an ANG II-antagonist which may possibly be used.

WO-97/37688 describes the combined use of HMG-CoA-reductase inhibitors and ANG II antagonists for the treatment of numerous conditions, including hypertension and atherosclerosis. Pravastatin, simvastatin, lovastatin and fluvastatin are mentioned as possible HMG-CoA-reductase inhibitors which may be used.

WO-99/11260 describes the combined use of a special HMG-CoA-reductase-inhibitor and ANG II antagonists for lowering blood pressure and the lipid levels and also for treating angina pectoris and atherosclerosis in mammals. The particular HMG-CoA-reductase-inhibitor is atorvastatin. Losartan, irbesartan and valsartan are mentioned as possible ANG II antagonists which are preferably used. Other ANG II antagonists mentioned are candesartan and eprosartan.

WO-00/45818 describes the combined use of an HMG-CoA-reductase-inhibitor and an ANG II antagonist for alleviating diabetic neuropathy and particularly for improving the conductive speed of the nerves and blood flow to the nerves in patients suffering from diabetes. The above examples of possible combinations are combinations comprising the statins pravastatin, simvastatin, cerivastatin, fluvastatin, atorvastatin and statin (E) together with the ANG II antagonists losartan, irbesartan, valsartan and candesartan, of which candesartan is preferred.

WO-01/15674 describes the combination of an inhibitor of the Renin-Angiotensin-System together with another antihypertensive, cholesterol-lowering agent, a diuretic or aspirin for preventing cardiovascular events such as stroke, congestive heart failure, cardiovascular death, myocardial infarct, worsening of angina, stoppage of the heart, revascularisation processes, diabetes and diabetic complications. Examples of possible combinations are the combinations of Angiotensin-Converting-Enzyme (ACE) inhibitors, i.e. compounds whose names end in “-pril”, such as captopril, imidapril, ramipril and the like, with the cholesterol level lowering agents lovastatin, pravastatin, simvastatin or fluvastatin.

### SUMMARY OF THE INVENTION

The present invention relates to a method for the prevention or treatment of cardiovascular, cardiopulmonary, pulmonary or renal diseases by improving endothelial function and achieving protection of organs, tissues and blood vessels in indications in which control of blood pressure and lipid levels are necessary, particularly in people in whom type 2 diabetes mellitus has been diagnosed or who are suspected of prediabetes, for preventing diabetes and prediabetes, or for the treatment of Metabolic Syndrome and insulin resistance in patients with normal blood pressure. The process comprises generally administering effective amounts of telmisartan or a polymorph or salt thereof and atorvastatin. The invention further relates to suitable pharmaceutical compositions which contain telmisartan or a polymorph or salt thereof and atorvastatin, as a combined preparation for simultaneous, separate or sequential use in the prevention or treatment of these diseases.

### DETAILED DESCRIPTION

Within the scope of the present invention it has now surprisingly been found that the angiotensin II receptor antagonist telmisartan and the salts thereof not only act to reduce blood pressure, in known manner, but are also capable of increasing the expression of genes in a cellular system, the transcription of which is known to be regulated by the PPARgamma receptor. In order to ensure comparable conditions this effect is observed and quantified within the scope of the present invention by means of a stably transformed cell line (cf. Example 2). The cells used are CHO cells which are the result of transformation with two gene constructs. The first of these constructs codes for the luciferase gene from Photinus pyralis (de Wet J R, Mol Cell Biol (1987) 7:725) under the control of a synthetic promoter with a five-fold repeat of a yeast Gal4 binding site (cf. GeneBank Sequence AF058756). The second construct codes for a fusion protein consisting of the ligand binding domain of the human PPARgamma2 transcription factor (cf. GeneBank Sequence U79012) and the yeast GAL4 DNA binding domain (Amino acids 1-147; Sadowski I, Nucleic Acids Res (1989) 17:7539).

The induction of the transcription of PPARgamma-regulated genes is known from the thiazolidinediones used as antidiabetic drugs (e.g. rosiglitazone) and is brought about by their binding to the PPARgamma Receptor and its activation. Within the scope of the test system used here this effect may be quantified as an induced luciferase activity of the transformed cell line. In the case of telmisartan, contrary to expectation, the same induction of a luciferase activity does not take place by the binding of the active substance to the PPARgamma Receptor. Binding of telmisartan to the PPARgamma receptor cannot be detected in various test systems. It is therefore presumed that the increase in the affinity of cofactor proteins for PPARgamma caused by the angiotensin II receptor antagonist telmisartan also leads to the recruiting of the cofactor proteins if there are no high-affinity synthetic PPARgamma ligands present. This then brings about activation of the transcription of genes regulated by the PPARgamma receptor, this activation being mediated by these cofactors. As the induction of these genes is responsible for the anti-diabetic activity of the thiazolidinediones it can be assumed that the induction of the same genes by telmisartan results in a comparable anti-diabetic activity. Thus, these active substances are suitable not only for treating high blood pressure but also for treating and preventing type 2 diabetes mellitus. This includes the treatment and prevention of metabolic syndrome, syndrome X or insulin-resistance syndrome.

The discovery of this new therapeutic effect of telmisartan and the salts thereof means that they can be used to produce a pharmaceutical composition for the treatment of people or mammals in whom the prevention or treatment of cardiovascular, cardiopulmonary, pulmonary or renal diseases is indicated, particularly if type 2 diabetes mellitus has been diagnosed or if there is a suspicion of prediabetes or who has been diagnosed with the metabolic disorder known as insulin resistance syndrome.

Of particular importance is the treatment of people in whom prevention or treatment of hypertension combined with hyperlipidaemia or atherosclerosis is indicated, or the treatment of asthma, bronchitis or interstitial lung diseases. They are also suitable for the treatment and prevention of type 2 diabetes and pre-type 2 diabetes. This includes the treatment and prevention of Metabolic Syndrome, Syndrome X, or Insulin Resistance Syndrome. Of particular importance is the treatment of people in whom prevention or treatment of hypertension combined with hyperlipidaemia or atherosclerosis is indicated, or the treatment of asthma, bronchitis or interstitial lung diseases.

Type 2 diabetes mellitus manifests itself in a fasting blood sugar level exceeding 125 mg of glucose per dl of plasma; the measurement of blood glucose values is a standard procedure in routine medical analysis. If a glucose tolerance test is carried out, the blood sugar level of a diabetic will be in excess of 200 mg of glucose per dl of plasma 2 hours after 75 g of glucose have been taken on an empty stomach. In a glucose tolerance test 75 g of glucose are administered orally to the patient being tested after 10-12 hours of fasting and the blood sugar level is recorded immediately before taking the glucose and 1 and 2 hours after taking it. In a healthy subject the blood sugar level before taking the glucose will be between 60 and 110 mg per dl of plasma, less than 200 mg per dl 1 hour after taking the glucose and less than 140 mg per dl after 2 hours. If after 2 hours the value is between 140 and 200 mg this is regarded as abnormal glucose tolerance.

If insulin resistance can be detected this is a particularly strong indication of the presence of prediabetes. Thus, it may be that in order to maintain glucose homoeostasis one person needs 2-3 times as much insulin as another person, without this having any direct pathological significance. The most certain method of determining insulin resistance is the euglycaemic-hyperinsulinaemic clamp test. The ratio of insulin to glucose is determined within the scope of a combined insulin-glucose infusion technique. There is found to be insulin resistance if the glucose absorption is below the 25th percentile of the background population investigated (WHO definition). Rather less laborious than the clamp test are so called minimal models in which, during an intravenous glucose tolerance test, the insulin and glucose concentrations in the blood are measured at fixed time intervals and from these the insulin resistance is calculated. Another method of measurement is the mathematical HOMA model. The insulin resistance is calculated by means of the fasting plasma glucose and the fasting insulin concentration. In this method it is not possible to distinguish between hepatic and peripheral insulin resistance. These processes are not really suitable for evaluating insulin resistance in daily practice. As a rule, other parameters are used in everyday clinical practice to assess insulin resistance. Preferably, the patient's triglyceride concentration is used, as increased triglyceride levels correlate significantly with the presence of insulin resistance.

Thus, there is a suspicion of prediabetes if the fasting blood sugar level is above the normal maximum level of 110 mg of glucose per dl of plasma but does not exceed the threshold of 125 mg of glucose per dl of plasma which indicates diabetes. Another indication of prediabetes is abnormal glucose tolerance, i.e. a blood sugar level of 140-200 mg of glucose per dl of plasma 2 hours after taking 75 g of glucose after a fast within the scope of a glucose tolerance test.

A triglyceride blood level of more than 150 mg/dl also indicates the presence of pre-diabetes. This suspicion is confirmed by a low blood level for HDL cholesterol. In women, levels below 40 mg per dl of plasma are regarded as too low while in men levels below 50 mg per dl of plasma are regarded as too low. Triglycerides and HDL cholesterol in the blood can also be determined by standard methods in medical analysis and are described for example in Thomas L (Editor): “Labor und Diagnose”, TH-Books Verlagsgesellschaft mbH, Frankfurt/Main, 2000. A suspicion of prediabetes is further confirmed if the fasting blood sugar levels also exceed 110 mg of glucose per dl of plasma. If the blood levels measured are in the region of these threshold values, the ratio of the waist measurement to the hip measurement can be used as an additional aid to make the decision. If this ratio exceeds a value of 0.8 in women or 1 in men, treatment is indicated.

Telmisartan is particularly indicated for treating diabetes or suspected prediabetes if hypertension also has to be treated. This is the case if the systolic blood pressure exceeds a value of 140 mm Hg and diastolic blood pressure exceeds a value of 90 mm Hg. If a patient is suffering from manifest diabetes it is currently recommended that the systolic blood pressure be reduced to a level below 130 mm Hg and the diastolic blood pressure be lowered to below 80 mm Hg. To achieve these levels it may be indicated in certain cases to combine angiotensin II receptor antagonists with a diuretic or a calcium antagonist. The term “diuretic” includes thiazides or thiazide analogues such as hydrochlorothiazides (HCTZ), clopamide, xipamide or chlorthalidone, aldosterone antagonists such as spironolactone or eplerenone and also other diuretics suitable for treating high blood pressure such as furosemide and piretanide, and combinations thereof with amiloride and triamterene.

The present invention means that for subjects being treated for increased blood pressure, the angiotensin II receptor antagonist telmisartan is indicated whenever the development of prediabetes is to be prevented or manifest diabetes is to be treated.

In only 10% of all cases of elevated blood pressure (secondary hypertension) is it possible to determine an identifiable cause such as e.g. kidney disease. As a rule, secondary hypertension can be remedied by treating and removing the cause. However, in almost 90% of all cases it is primary hypertension, the exact cause of which is not known and which therefore cannot be directly cured. The negative effects of elevated blood pressure can be reduced by changing lifestyle and correct treatment. The interaction of different risk factors or the combined occurrence of individual risk factors appear to cause high blood pressure. In particular, the combination of high blood pressure with disorders of the fat and sugar metabolism is observed to an increasing extent. These disorders are often unnoticed to begin with but can be recognised from increased blood levels of triglycerides and glucose and lower blood levels of HDL cholesterol. At a fairly advanced stage they can also be detected in slowly increasing corpulence. These disorders can be explained by increasing insulin resistance. The less effective the insulin, the more the fat and sugar metabolisms are disrupted. The combination of all these disorders in the last analysis increases the probability of contracting the sugar disease diabetes and dying prematurely of heart or vascular disease.

As primary or essential hypertension is a multifactorial disease, it seems unlikely that insulin resistance or hyerinsulinaemia is the sole cause of high blood pressure. A number of observations indicate, however, that defects in the insulin metabolism have a hypertensive effect and thus predispose to high blood pressure. In connection with this, reference may be made to hypertensive insulin resistance. Thus the presence of insulin resistance can be detected in about 50% of normal-weight hypertensives and normotensive close relatives. In obese patients not only is there a higher level of insulin resistance, but also a stronger correlation between hypertension and hyperinsulinaemia than in slim hypertensives.

Estimates are based on the supposition that about a third of adults in those parts of the world with an excessive supply of food are affected by the combination of high blood pressure and disorders of the fat and sugar metabolism and that this number will continue to increase. Consequently there is a need for drugs which are capable of helping to slow down or stop the progress of the above-mentioned metabolic disorders at the earliest possible stage and at the same time to obviate the detrimental effects of increased blood pressure on the health.

The present invention now discloses a pharmaceutical composition which can be used both to treat hypertension and hyperlipidaemia simultaneously and to treat manifest type 2 diabetes or the first signs of the complex metabolic disorder of prediabetes. The new active substance combination is particularly suitable for the treatment and prevention of the above-mentioned hypertensive insulin resistance, which denotes insufficient utilisation of the insulin circulating in the bloodstream, combined with a resulting increase in blood pressure. Thus, the invention also includes diabetes prevention in patients who are being treated for high blood pressure and hyperlipidaemia. If therefore the combination of telmisartan and atorvastatin is used immediately to control blood pressure, hyperlipidaemia or hypertensive insulin resistance as soon as one of the above-mentioned signs of prediabetes is present, the onset of manifest type 2 diabetes can be delayed or prevented.

Telmisartan and the suitable salts thereof thus:

do not exhibit any in vitro binding to the ligand binding domain of a human PPARgamma receptor, but lead to the

induction of a luciferase activity when they are added to the culture medium of a stably transformed PPARgamma reporter cell line which

a) expresses a fusion protein consisting of the ligand binding domain of the human PPARgamma transcription factor and the yeast GAL4 DNA binding domain and

b) a luciferase gene under the control of a five-times repeated yeast Gal4 binding site.

The preparation of a PPARgamma reporter cell line of this kind is described in Example 2.

There is no in vitro binding to the ligand binding domain of the human PPARgamma2 receptor if it cannot be detected in an AlphaScreen (Ullmann E F et al, Proc Natl Acad Sci USA (1994) 91:5426-5430). Instead of an Alpha Screen, an SPA assay (Mukherjee R et al., J Steroid Biochem Mol Biol (2002) 81:217-225) or an NMR investigation (Johnson B A et al., J Mol Biol (2000) 298:187-194) may also be carried out. As a rule, binding to the receptor cannot be detected by any of these methods.

If it appears useful or necessary to use an angiotensin II receptor blocker in conjunction with one or more other therapeutic active substances, telmisartan is a preferred angiotensin II receptor blocker, as it combines a blood pressure lowering and antidiabetic activity in a single active substance and helps to prevent diabetes. For this reason, preformulated active substance combinations of telmisartan with the HMG-Co A reductase inhibitor atorvastatin constitute a major further development in the treatment of cardiovascular, cardiopulmonary, pulmonary or renal diseases, but particularly when there is a need to treat hyperlipidaemia, prediabetes or manifest type 2 diabetes, osteoporosis or Alzheimer's simultaneously, as well as prevent diabetes.

It is observed that by joint administration of an effective amount of telmisartan with an effective amount of atorvastatin, or polymorphs or salts thereof, surprising advantages can be achieved in the prevention or treatment of cardiovascular, cardiopulmonary, pulmonary or renal diseases in patients requiring treatment with a high degree of efficacy, irrespective of the known hypotensive effect of the active substance telmisartan and independently of the antihyperlipidaemic activity of the active substance atorvastatin, compared with administering the ANG II antagonist or the HMG-CoA-reductase inhibitor on its own. Thus, it is possible for example to control the expression of the Matrix Metalloproteinase MMP-9, which is expressed to a greater extent in chronic inflammation of the respiratory tract or in type 2 diabetes. Elevated plasma levels of the inflammation-promoting cytokine CD40L can also be counteracted. Increased plasma levels of CD40L are a known risk factor for cardiovascular diseases.

It is also observed that the prevention or treatment improves endothelial function and affords protection of organs, tissues and blood vessels in diseases in which there is a need to control both blood pressure and also the lipid levels. Thus, the elasticity of the arteries can be improved and in the skin an enhanced production of NO, a marker of endothelial function, can be achieved.

It is also observed that the prevention or treatment is particularly effective in the following situations:

indications (A) which can be positively influenced by inhibition of the activities mediated by the AT1 -receptor and maintenance of the activities of angiotensin II (ANG II) mediated by the AT2-receptor and by inhibition of the HMG-CoA-reductase activities, by means of which the activities mediated by bradykinin can thus be potentiated and antihyperlipidaemic activities can be achieved; or

indications (B) which go hand in hand with an increase in the AT1 receptors in the subepithelial region or an increase in the AT2 receptors in the epithelium.

Suitable indications (A) are selected from the following indications:

treatment of combined hypertension and hyperlipidaemia;

reduced occurrence of stroke, acute myocardial infarct or cardiovascular deaths, particularly in people with an increased risk of adverse cardiovascular events or strokes;

provision of a renoprotective effect, e.g. in renal failure or diabetic nephropathy;

prevention of left ventricular hypertrophy, vascular thickening, e.g. prevention of the thickening of blood vessel walls after vascular surgery, improvement of the chances of survival after heart transplants, prevention of arterial restenosis after angioplasty, prevention or treatment of atherogenic disorders such as atherosclerosis, protection against coronary artery diseases, prevention of atheroma progression and prevention of diabetic angiopathy;

lowering of cholesterol, lowering of plasma-fibrinogen and plasma viscosity, inhibition of the proliferation of smooth muscle cells, reduction of the ability of macrophages to oxidise LDL, protection of heart muscle cells from hypoxic damage and lowering of the plasminogen activator inhibitor 1 (PAI-1);

prevention or treatment of ischaemic peripheral circulatory disorders and myocardial ischaemia (angina); and

prevention of the progression of heart failure after myocardial infarct.

Suitable indications (B) are selected from the following indications: obstructive respiratory diseases, chronic obstructive lung diseases such as bronchitis or chronic bronchitis, emphysema, for example caused by asthma, cystic fibrosis, interstitial lung disease, lung cancer, pulmonary vascular diseases and increased resistance to the airflow in forced ventilation; adult respiratory distress syndrome (ARDS), reduction in the proliferative capacity of the epithelium in cancer of the lung and breast, treatment of sepsis syndromes, lung damage such as inflammation of the lung, aspiration of the stomach contents, trauma to the ribcage, shock, burns, fatty embolisms, heart-lung bypass, O₂ toxicity, haemorrhagic pancreatitis, interstitial and bronchoalveolar inflammation, particularly when accompanied by increased expression of Matrix Metalloproteinase such as MMP-9, proliferation of epithelial and interstitial cells, collagen accumulation and fibrosis.

Thus, the present invention provides a process for the prevention or treatment of hypertension and hyperlipidaemia, particularly in a mammal in whom diabetes has been diagnosed or there is a suspicion of prediabetes, the process comprising the combined administration of an effective amount of the HMG-CoA-reductase-inhibitor atorvastatin or a polymorph or salt thereof, together with an effective amount of the ANG II antagonist telmisartan or a polymorph or salt thereof.

The invention further relates to the combined use of telmisartan and atorvastatin or the combined use of polymorphs or salts of these active substances in the manufacture of a pharmaceutical composition for the prevention or treatment of hypertension in combination with hyperlipidaemia, particularly if diabetes has been diagnosed or there is a suspicion of prediabetes.

Thus, the advantageous activity of the processes according to the invention is based primarily on the protective effective of the combined treatment for organs, tissues and blood vessels, as well as the preventive effect in relation to diabetes.

The above-mentioned unexpected advantages may be attributable to a more effective blockade of the activities of ANG II mediated by the AT1 receptor, to the activity of ANG II mediated by the AT2 receptor, which remains unaffected by this specific ANG II antagonist, together with an increase in the activities mediated by bradykinin, to the PPARgamma-like transcription activation and to the achievement of an antihyperlipidaemic activity by atorvastatin.

It is observed, for example, that the combined administration of the specific ANG II antagonist telmisartan with the specific HMG-CoA-reductase-inhibitor atorvastatin or the combined administration of polymorphs or salts of these active substances brings about a significant prevention of cardiovascular deaths and overall mortality, particularly in respect of the occurrence of stroke and acute myocardial infarct, compared with the administration of one of these active substances on its own.

Therefore, a preferred process according to the invention comprises reducing the occurrence of stroke and acute myocardial infarct in people or non-human mammals requiring treatment, particularly in individuals with manifest type 2 diabetes or suspected prediabetes or with a increased risk of adverse cardiovascular events or stroke, by administering telmisartan together with atorvastatin or by administering polymorphs or salts of these active substances together.

It is observed, moreover, that the combined treatment and the corresponding compositions which specifically contain an amount of the HMG-CoA-reductase inhibitor atorvastatin together with an amount of the ANG II antagonist telmisartan or polymorphs or salts of these active substances, result in a high activity in the regulation of blood pressure and in lipid regulation in mammals. It is expected that the synergistic activity achieved using this special combination is surprisingly superior to the activity of corresponding conventional combinations.

By a synergistic combination for regulating blood pressure and lipids is meant that it contains an amount of atorvastatin and an amount of telmisartan or polymorphs or salts of these active substances, wherein the quantity of the individual active substance is not sufficient on its own to achieve the therapeutic effect which is obtained by administering the combination of agents, and the combined effects of the quantities of therapeutic agents are greater than the sum of the therapeutic activities which can be achieved with the quantities of the individual therapeutic agents.

The present invention further relates to pharmaceutical compositions containing telmisartan or one of the salts thereof combined with atorvastatin and the preparation thereof. The pharmaceutical compositions are used for treating human or non-human mammals for the prevention or treatment of the above-mentioned diseases or indications and contain telmisartan and atorvastatin, optionally together with pharmaceutically acceptable diluents and/or carriers, in the form of a combined preparation for simultaneous, separate or successive use in the prevention or treatment of these diseases or indications.

These combinations of active substances are generally incorporated with one or more formulation adjuvants such as mannitol, sorbitol, xylitol, saccharose, calcium carbonate, calcium phosphate, lactose, croscarmellose sodium salt (cellulose carboxymethylether sodium salt, cross-linked), crospovidone, sodium starch glycolate, hydroxypropylcellulose (low-substituted), maize starch, polyvinylpyrrolidone, copolymers of vinylpyrrolidone with other vinyl derivatives (copovidone), hydroxypropylcellulose, hydroxypropylmethylcellulose, microcrystalline cellulose or starch, magnesium stearate, sodium stearylfumarate, talc, hydroxypropylmethylcellulose, carboxymethylcellulose, cellulose acetate phthalate, polyvinyl acetate, water, water/ethanol, water/glycerol, water/sorbitol, water/polyethyleneglycol, propyleneglycol, cetylstearyl alcohol, carboxymethylcellulose or fatty substances such as hard fat or suitable mixtures thereof, into conventional galenic preparations such as plain or coated tablets, capsules, powders, suspensions or suppositories.

Tablets may be obtained for example by mixing the active substance or substances with one or more excipients and subsequently compressing them. The tablets may also consist of several layers.

Examples of excipients are:

inert diluents such as mannitol, sorbitol, xylitol, saccharose, calcium carbonate, calcium phosphate and lactose;

disintegrants such as croscarmellose sodium salt (cellulose carboxymethylether sodium salt, cross-linked), crospovidone, sodium starch glycolate, hydroxypropylcellulose (low-substituted) and maize starch;

binders such as polyvinylpyrrolidone, copolymers of vinylpyrrolidone with other vinyl derivatives (copovidone), hydroxypropylcellulose, hydroxypropylmethylcellulose, microcrystalline cellulose or starch;

lubricants such as magnesium stearate, sodium stearyl fumarate and talc;

agents for achieving delayed release such as hydroxypropylmethylcellulose, carboxymethylcellulose, cellulose acetate phthalate and polyvinyl acetate; and

pharmaceutically permitted colourings such as coloured iron oxides.

In all aspects of the present invention the particular ANG II antagonist telmisartan is {4′-[2-n-propyl-4-methyl-6-(1-methylbenzimidazol-2-yl)-benzimidazol-1-ylmethyl]-biphenyl-2-carboxylic acid} or polymorphs or salts thereof, preferably the sodium salt. Telmisartan is already on the market, e.g. under the name Micardis®.

Telmisartan is described for example in EP-0 502 314 and U.S. Pat. No. 5,591,762. Polymorphs of telmisartan are described for example in WO-00/43370, U.S. Pat. No. 6,358,986 and U.S. Pat. No. 6,410,742. Sodium salts of telmisartan are described for example in WO 03/037876.

For example it states in WO 03/037876 that the sodium salt of telmisartan of formula:

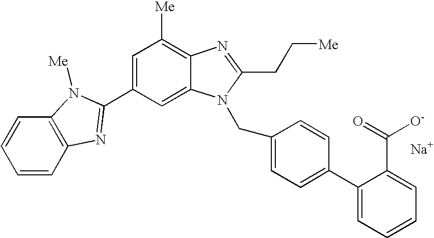

can be selectively obtained in a crystalline polymorphic form by a suitable choice of the manufacturing conditions.

This crystalline form of the sodium salt of telmisartan is characterised by the melting point T=245±5° C. (determined by differential scanning calorimetry using the Mettler-Toledo DSC82 apparatus; heating rate: 10° K/min).

The sodium salt of telmisartan may be prepared using one of the following two manufacturing processes.

According to all aspects of the invention the HMG-CoA-reductase inhibitor is atorvastatin or polymorphs or salts thereof, preferably the hemicalcium salt {[R-(R*, R*)]-2-(4-fluorophenyl)β,δ-dihydroxy-5-(1-methylethyl)-3-phenyl-4-[(phenylamino)-carbonyl]-1H-pyrrol-1-heptanoic acid hemicalcium salt), which is marketed for example under the brand names Lipitor®, Zarator® and Sortis®.

Atorvastatin is described for example in EP 0 247 633 and U.S. Pat. No. 4,681,893. Polymorphs of atorvastatin are described for example in WO-97/03958, WO-97/03959, EP-0 848 704 and EP-1 148 049. Salts of atorvastatin (monopotassium salt, monosodium salt, calcium salt, magnesium salt, zinc salt and meglumine) are described for example in EP-0 409 281 and U.S. Pat. No. 5,273,995.

By combined administration of the two active substances is meant a successive or simultaneous administration, of which simultaneous administration is preferred.

For successive administration telmisartan may be given before or after the administration of atorvastatin.

The active substances may be administered by oral, buccal or parenteral route, by inhalation, or rectally or topically; oral administration is preferred. Parenteral administration may comprise subcutaneous, intravenous, intramuscular and intrasternal injections as well as infusion techniques.

The active substances may be given orally in a variety of different dosage forms, i.e. they may be prepared with various pharmaceutically acceptable inert carriers to form tablets, capsules, pastilles, sweets, powders, sprays, aqueous suspensions, elixirs, syrups and the like. Such carriers include solid diluents or fillers, sterile aqueous media and various non-toxic organic solvents. In addition, oral pharmaceutical preparations of this kind may be provided with suitable sweeteners and/or flavourings, using various agents conventionally used for such purposes. In general the compounds according to the invention are present in oral formulations of this kind in concentrations ranging from about 0.5 to about 90 wt %, based on the total composition, in amounts such that they produce the desired dosage units. Other suitable dosage forms for the compounds according to the invention include preparations and devices with controlled release, with which the skilled man will be familiar.

For oral administration it is possible to use tablets containing various carriers such as sodium citrate, calcium carbonate and calcium phosphate together with various disintegrants, such as starch and preferably potato or tapioca starch, alginic acid and certain complex silicates, together with binders such as polyvinylpyrrolidone, saccharose, gelatine and gum arabic. Lubricants such as magnesium stearate, sodium laurylsulphate and talc or compositions of a similar type may also be used as fillers in filled soft and hard gelatine capsules. These may also include lactose or milk sugar as well as high molecular polyethyleneglycols. If aqueous suspensions and/or elixirs are desired for oral administration, the active substances may be combined with various sweeteners or flavourings, colouring agents or dyes and optionally emulsifiers and/or water, ethanol, propyleneglycol, glycerol and various combinations thereof.

For parenteral administration solutions of the compounds in sesame or groundnut oil or in aqueous propyleneglycol as well as sterile aqueous solutions of the corresponding pharmaceutically acceptable salts may be used. Aqueous solutions of this kind may optionally be suitably buffered and the liquid diluent may optionally be made isotonic with sufficient quantities of common salt or glucose. These special aqueous solutions are particularly suitable for intravenous, intramuscular and subcutaneous injection. Sterile aqueous media may easily be obtained by conventional methods known to the skilled man. For example, distilled water is normally used as a liquid diluent. The finished preparation is passed through a suitable bacterial filter, e.g. a filter made of sintered glass, diatomaceous earth or unglazed porcelain. Preferred filters of this type include Berkefeld, Chamberland and asbestos disc metal Seitz filters, the liquid being aspirated into a sterile container using a suction pump. Throughout the entire process of preparing these injectable solutions the necessary steps should be carried out in such a way as to obtain the end products in a sterile state.

For transdermal administration the formulations of the special compounds or combinations include for example solutions, lotions, ointments, creams, gels, suppositories, delayed-release preparations and devices therefor. These formulations comprise the compound(s) in particular and may contain ethanol, water, penetration promoters and inert carriers, e.g. gel-forming materials, mineral oil, emulsifiers, benzyl alcohol and the like.

The formulations prepared contain, for example, an equivalent of 2.5-40 mg, preferably 5, 10, 15, 20, 25, 30, 35 or 40 mg of atorvastatin. Atorvastatin or polymorphs or salts thereof may be administered in daily doses of about 1.25 mg (or 0.018 mg/kg of body weight, based on a person weighing 70 kg) to about 450 mg (6.43 mg/kg of body weight, based on a person weighing 70 kg) by oral route, about 20 mg (0.286 mg/kg of body weight, based on a person weighing 70 kg) by parenteral route and preferably in a dosage of about 2.5 mg (0.036 mg/kg of body weight, based on a person weighing 70 kg) to about 80 mg (1.428 mg/kg of body weight, based on a person weighing 70 kg) by oral route. Particularly preferred is an oral daily dose of about 5 mg (0.071 mg/kg of body weight, based on a person weighing 70 kg), about 10 mg (0.143 mg/kg of body weight, based on a person weighing 70 kg), about 20 mg (0.286 mg/kg of body weight, based on a person weighing 70 kg) or about 40 mg (0.571 mg/kg of body weight, based on a person weighing 70 kg) or, especially to start with, an oral daily dose of about 10 mg by oral route.

The formulations prepared contain, for example, an equivalent of 20-200 mg, preferably 20, 40, 80, 120, 160 or 200 mg of the free acid of telmisartan. If the active substance is combined with hydrochlorothiazide (HCTZ) or clorthalidone, the formulation contains 10-50 mg, preferably 50, 25 or 12.5 mg of the diuretic. Telmisartan or polymorphs or salts thereof may be administered in a daily dose of 10 mg (or 0.143 mg/kg of body weight, based on a person weighing 70 kg) to 500 mg (7.143 mg/kg of body weight, based on a person weighing 70 kg) by oral route and about 20 mg (0.286 mg/kg of body weight, based on a person weighing 70 kg) by parenteral route, preferably 20 mg (0.286 mg/kg of body weight, based on a person weighing 70 kg) to 100 mg (1.429 mg/kg of body weight, based on a person weighing 70 kg) by oral route. Particularly preferred is an oral daily dose of 40 mg (0.571 mg/kg of body weight, based on a person weighing 70 kg) to 80 mg (1.143 mg/kg of body weight, based on a person weighing 70 kg) or in particular a dose of about 80 mg (1.143 mg/kg of body weight, based on a person weighing 70 kg).

Preferably the ratio of atorvastatin to telmisartan or the polymorphs or salts thereof in the pharmaceutical combination is 1:100 to 100:1 (based on weight).

In particularly preferred embodiments atorvastatin or a polymorph or salt thereof together with telmisartan or a polymorph or salt thereof is administered by oral route in the following daily doses:

10 mg of atorvastatin and 40 mg of telmisartan (or polymorphs or salts thereof);

10 mg of atorvastatin and 80 mg of telmisartan (or polymorphs or salts thereof);

20 mg of atorvastatin and 40 mg of telmisartan (or polymorphs or salts thereof);

20 mg of atorvastatin and 80 mg of telmisartan (or polymorphs or salts thereof).

According to a preferred embodiment the pharmaceutical compositions according to the invention contain the HMG-CoA-reductase-inhibitor in an amount of 1.25 mg to 450 mg and the ANG II antagonist in an amount of 10 mg to 500 mg in individual dosage units, optionally together with one or more pharmaceutically acceptable diluents and/or carriers.

According to another preferred embodiment the pharmaceutical compositions according to the invention contain atorvastatin in an amount of 2.5 mg to 80 mg and telmisartan in an amount of 20 to 100 mg in individual dosage units, optionally together with one or more pharmaceutically acceptable diluents and/or carriers.

Another preferred sub-group of pharmaceutical compositions according to the invention contain atorvastatin in an amount of 5 mg to 20 mg and telmisartan in an amount of 40 mg to 80 mg in individual dosage units, optionally together with one or more pharmaceutically acceptable diluents and/or carriers.

Another preferred sub-group of pharmaceutical compositions according to the invention contain atorvastatin in an amount of 10 or 20 mg and telmisartan in an amount of 40 or 80 mg in individual dosage units, optionally together with one or more pharmaceutically acceptable diluents and/or carriers.

As already mentioned, the present invention also relates to the use of telmisartan for preparing a pharmaceutical composition for treating the human or non-human mammalian body for the prevention or treatment of the above-mentioned indications when used in combination with atorvastatin. By this use is meant the preparation of all the above-mentioned pharmaceutical compositions according to the invention.

### EXAMPLES

### Example 1

### Telmisartan, Losartan and Irbesartan do not Bind in vitro to the PPARgamma Ligand Binding Domain

Protein containing the human PPARgamma-ligand binding domain (LBD) was prepared as a GST fusion protein in E. coli and purified by affinity chromatography. To do this, a DNA section which codes for the amino acids 205-505 of the human PPARgamma2 transcription factor (cf. Genbank entry U79012) was subcloned via the additionally inserted restriction cutting sites BamH I and Xho I into the expression vector pGEX-4T-1 (Amersham) and the sequence of the section was monitored. The fusion protein was expressed in the E. coli strain BL21(DE3) recommended for pGEX vectors after induction with 0.2 mM IPTG for 4 hours at 25° C. The bacteria were pelleted after the induction and frozen in batches in PBS, pH 7.4. After opening up in a French Press, the dissolved GST-PPARgamma-LBD-fusion protein was purified using a GSTrap column (Pharmacia). Elution was carried out by the addition of 20 mM reduced glutathione. The GST-PPARgamma-LBD-protein fractions were desalinated using a HiTrap desalting column (Pharmacia) and the protein concentration was determined using a standard assay.

Protein containing the human RXRalpha ligand binding domain (LBD) was prepared as a His tag fusion protein in E. coli and purified by affinity chromatography. To do this a DNA section which codes for the amino acids 220-461 of the human RXRalpha transcription factor (cf. Genbank entry NM_{—}002957, nt 729-1457) was subcloned via the additionally introduced restriction cutting sites BamH I and Not I into the expression vector pET28c (Novagen) and the sequence of the section was monitored. The fusion protein was expressed in the E. coli strain BL21(DE3) recommended for pET vectors after induction with 0.2 mM IPTG for 4 hours at 25° C. The bacteria were pelleted after the expression and frozen in batches in PBS, pH 7.4. After opening up in a French Press, the dissolved His-RXRalpha-LBD-fusion protein is purified using a HiTrap chelating column (Pharmacia). Elution was carried out using a 500 mM imidazole step. The His-RXRalpha-LBD protein fractions were desalinated using a HiTrap desalting column (Pharmacia) and the protein concentration was determined using a standard assay.

a) AlphaScreen

Alpha Screen assays were first described in Ullmann E F et al., Proc Natl Acad Sci USA (1994) 91:5426-5430. The measurements carried out within the scope of this Example were carried out as described by Glickman J F et al., J Biomol Screen (2002) 7:3-10. The assay buffer consisted of 25mM Hepes pH7.4, 100 mM NaCl, 1 mM DTT, 0.1% Tween-20, 0.1% BSA. 3 nM GST-PPARgamma-LBD fusion protein, 15 nM biotinylated LXXLL peptide of the cofactor CBP (corresponding to the peptide disclosed on page 218 of Mukherjee R et al., J Steroid Biochem Mol Biol (2002) 81:217-225 with an additional N-terminal cysteine), and in each case 10 μg/ml of anti-GST-acceptor beads or streptavidine donor beads (Applied Biosystems) were incubated in a total volume of 12.5 μl in the presence of different concentrations of a test substance (in DMSO) for 4 hours at ambient temperature. The final DMSO concentration in the assay was 1% (v/v). A 1% DMSO solution was used as the background control (NSB). The measurement was done using a Packard fusion measuring device.

| | |
|---|---|
| telmisartan | rosiglitazon |

| conc./M | MW | SD | MW | SD |
|---|---|---|---|---|
| NSB | 619 | 21 | 573 | 17 |
| 1.00E−08 | | | 820 | 18 |
| 3.00E−08 | 642 | 41 | 1720 | 48 |
| 1.00E−07 | 606 | 10 | 8704 | 59 |
| 3.00E−07 | 644 | 56 | 27176 | 1232 |
| 1.00E−06 | 677 | 14 | 43233 | 1083 |
| 3.00E−06 | 720 | 35 | 52691 | 3771 |
| 1.00E−05 | 847 | 82 | 56366 | 4303 |
| 5.00E−05 | 1111 | 135 | | |

Unlike rosiglitazone, a PPARgamma-agonist known from the literature with binding in the LBD, the use of increasing concentrations of telmisartan, losartan and irbesartan (concentrations of up to 50 μM) did not result in any direct activation of the PPARgamma-LBD and hence in any significant recruiting of the LXXLL peptide.

b) SPA Assay

A description of the SPA assay format can be found in Mukherjee R et al., J Steroid Biochem Mol Biol (2002) 81:217-225. The assay buffer consisted of 20 mM Tris pH 7.5, 25 mM KCl, 10 mM DTT, 0.2% Triton X-100). Thirty (30)nM GST-PPARgamma-LBD fusion protein, 30 nM His-RXRalpha-LBD, anti-GST-antibody (1:600, Amersham Pharmacia), 0.25 mg protein A SPA PVT antibody-binding beads (Amersham Pharmacia), 30 nM ³H-labelled rosiglitazone were incubated with dilutions of the test substance for 5 hours at room temperature in a total volume of 100 μl.

10 μM of unlabelled rosiglitazone was added as background control (NSB) instead of the radioactive rosiglitazone, and the solvent used, e.g. DMSO, was added as the maximum value (Bmax) instead of a test substance.

After the incubation the test preparations were centrifuged for 5 minutes at 2000 rpm in a Hettich Universal 30Rf centrifuge and measured using a Packard TopCount NXT.

| | | |
|---|---|---|
| telmisartan | irbesartan | losartan |

| conc/M | MW | SD | MW | SD | MW | SD |
|---|---|---|---|---|---|---|
| NSB | 217 | 9 | 217 | 9 | 217 | 9 |
| Bmax | 911 | 15 | 911 | 15 | 911 | 15 |
| 1.00E−07 | 837 | 49 | 913 | 54 | 915 | 43 |
| 3.00E−07 | 802 | 28 | 810 | 49 | 835 | 11 |
| 1.00E−06 | 818 | 27 | 815 | 51 | 901 | 10 |
| 3.00E−06 | 818 | 20 | 779 | 26 | 814 | 53 |
| 1.00E−05 | 703 | 30 | 723 | 37 | 787 | 46 |
| 3.00E−05 | 691 | 222 | 648 | 40 | 784 | 96 |
| 1.00E−04 | 545 | 18 | 510 | 81 | 611 | 17 |

In contrast to direct PPARgamma-agonists which bind to the PPARgamma-LBD, no concentration-dependent displacement of the radioactive rosiglitazone from the binding pocket took place even in the presence of very large excesses of telmisartan, losartan or irbesartan.

c) NMR Investigations

In contrast to a direct PPARgamma ligand, e.g. rosiglitazone, no interaction of the test substance with amino acids in the binding pocket takes place during the measurement of the ¹⁵N TROSY spectrum of the PPARgamma-LBD in the presence of the test substance telmisartan. The amino acids of the binding pocket have the same position in the presence of the test substances as in the absence of a ligand.

### Example 2

### Preparation of a Stably Transformed PPARgamma Reporter Cell Line

A DNA section which codes for amino acids 205-505 of the human PPARgamma2 transcription factor (corresponding to nucleotides 703-1605 of Genbank sequence U79012) was incorporated into the Multiple Cloning Site of the vector pFA-CMV (Stratagene) via additionally inserted BamH I and Hind III restriction cutting sites and the sequence was verified. The resulting plasmid pFA-CMV/hPPARgamma2-LBD codes N-terminally of the PPARgamma-LBD in the same reading frame for a Gal4 DNA binding domain. In addition the plasmid codes for neomycin resistance.

The cell line CHO-K1 (ATCC CCL-61) was cotransfected with the plasmids pFA-CMV/hPPARgamma2-LBD and pFR-Luc (Stratagene). pFR-Luc codes for the luciferase gene under the control of a five-times repeated yeast Gal4 binding site. The transfection was carried out with lipofectamine2000 in accordance with the manufacturer's instructions.

After transfection the cells were cultivated in medium (Ham's F12 with 10% foetal calf serum) in the presence of 0.5 mg/ml G-418. After six days' cultivation the cells were passaged and kept in culture for another 10 days. The resulting neomycin-resistant colonies were picked out under the microscope and transferred into 96 well dishes and cultured. Various transformed cell lines were obtained with the plasmids contained therein (e.g. clone no. 10, 11, 13 etc), which were kept in the culture medium.

The cell lines were examined for the inducibility of the luciferase gene using a PPARgamma agonist, e.g. rosiglitazone, and react with an increased luciferase signal to stimulation by the PPARgamma agonist.

### Example 3

### Telmisartan, Losartan and Irbesartan Activate PPARgamma at Cellular Level

The CHO-K1 cell line derived from the transformed clone 11 of Example 2 was seeded in 96-well flat-bottomed dishes in a density of 3×10⁴ cells/200 μl/well and cultivated overnight in Ham's F-12 medium with 10% foetal calf serum and 0.5 mg/ml G-418. After 24 hours the medium is changed for one without any added G-418.

The test substances were brought to 100 times the desired concentration with a suitable solvent, e.g. DMSO, and diluted 1:100 with the medium placed in the cell culture plate. The solvent used, e.g. DMSO, was used as the background control in the same concentration.

24 hours after the addition of the substance the supernatants were discarded and the cells were washed twice with 150 μl washing buffer (25 mM Tricine, 16.3 mM MgSO₄, pH7.8). After the washing steps 50 μl of washing buffer with 150 μl of luciferase assay buffer (25 mM Tricine, 0.5 mM EDTA, 0.54 mM NaTPP, 16.3 mM MgSO₄, 1.2 mM ATP, 0.05 mM luciferine, 56.8 mM 2-mercaptoethanol, 0.1% Trition X-100, pH7.8) was added to each test preparation. Luminescence was measured after a five minute wait using a Packard TopCount NXT. The luciferase activity was obtained by integrating the relative luciferase units (RLU) of the first ten seconds after the start of measurement.

| | telmisartan | | irbesartan | | losartan | | rosiglitazone | |
|---|---|---|---|---|---|---|---|---|
| conc/M | MW | SD | MW | SD | MW | SD | MW | SD |
| NSB | 466 | 188 | 466 | 188 | 466 | 188 | 741 | 141 |
| 1.00E−08 | | | | | | | 2761 | 178 |
| 3.00E−08 | | | | | | | 8256 | 708 |
| 1.00E−07 | | | | | | | 35265 | 2947 |
| 3.00E−07 | 760 | 255 | 491 | 70 | 874 | 475 | 86859 | 6139 |
| 1.00E−06 | 2859 | 455 | 657 | 65 | 589 | 70 | 106252 | 30018 |
| 3.00E−06 | 24498 | 2290 | 1028 | 342 | 672 | 88 | 143232 | 14064 |
| 1.00E−05 | 61397 | 7853 | 3292 | 556 | 709 | 163 | 150989 | 24245 |
| 3.00E−05 | 58790 | 2055 | 22133 | 4202 | 3271 | 585 | | |
| 1.00E−04 | | | 29600 | 6936 | 11322 | 1668 | | |

The angiotensin II receptor antagonist telmisartan brought about a particularly potent activation of the PPARgamma pathway in the PPARgamma reporter cell line. Activation by other angiotensin II receptor antagonists such as losartan and irbesartan took place only at higher test concentrations and to a lesser extent.

### Example 4

### Examples of Formulations

Tablet 1

Tablets having the following composition were obtained by direct compression of the telmisartan sodium salt with excipients and magnesium stearate:

| Ingredients: | mg |
|---|---|
| telmisartan sodium salt | 41.708 |
| mannitol | 49.542 |
| microcrystalline cellulose | 50.000 |
| croscarmellose sodium salt | 5.000 |
| magnesium stearate | 3.750 |
| total | 250.000 |

Tablet 2

Tablets having the following composition were obtained by direct compression of the telmisartan sodium salt with excipients and magnesium stearate:

| Ingredients: | mg |
|---|---|
| telmisartan sodium salt | 83.417 |
| sorbitol | 384.083 |
| polyvidone K25 | 25.000 |
| magnesium stearate | 7.500 |
| total | 500.000 |

Tablet 3

Hydrochlorothiazide, telmisartan sodium salt, sorbitol and red iron oxide were mixed in a free fall blender, passed through a 0.8 mm screen and, after the addition of magnesium stearate, processed in a free fall blender to obtain a powdered mixture.

This combination of active substances and excipients was then compressed with a suitable tablet press (e.g. Korsch EK0 or Fette P1200) to form tablets. Tablets with the following composition were obtained, the quantity of telmisartan sodium salt contained in each tablet corresponding to a quantity of 80 mg of the free acid of telmisartan.

| Ingredient | mg/tablet | % |
|---|---|---|
| telmisartan sodium salt | 83.417 | 13.903 |
| hydrochlorothiazide | 12.500 | 2.083 |
| sorbitol | 494.483 | 82.414 |
| red iron oxide | 0.600 | 0.100 |
| magnesium stearate | 9.000 | 1.500 |
| total | 600.000 | 100.000 |

The telmisartan sodium salt of the tablets of the three batches dissolved in 900 ml of 0.1 M phosphate buffer, pH 7.5, at a rate of 92±1.5%, 96±1.8% and 100±1.0%, respectively, after 30 minutes' stirring (75 rpm). The hydrochlorothiazide dissolved in 900 ml of 0.1 M HCl (100 rpm) after 30 minutes at a rate of 69±6.3%, 72±2.1% and 78±1.8%, respectively.

### Example 5

### Preparation of a Crystalline Telmisartan Sodium Salt, Starting from Telmisartan

The starting material for preparing crystalline telmisartan sodium salt may be the free acid of telmisartan, which may be obtained by conventional methods (e.g. according to EP-0 502 314).

154.4 g of telmisartan were placed in 308.8 ml of toluene in a suitable reaction vessel, the suspension was combined with 27.8 g of a 44.68% sodium hydroxide solution and 84.9 ml of ethanol and heated to 78° C. for about 30 minutes. Then the mixture was filtered. If large amounts of solid were left in the filter, the filter was, optionally, washed with a mixture of 61.8 ml of toluene and 15.3 ml of ethanol.

463.2 ml of toluene were placed in another reaction vessel and refluxed. The filtrate obtained according to the process described above was slowly added at the boiling temperature and simultaneously distilled azeotropically. After it was all added, any solution obtained by washing the filter was also added and again azeotropic distillation was carried out. The mixture was distilled until a temperature of 103° C. was obtained. Then the suspension was cooled to ambient temperature. The crystals were suction filtered, washed with 154.4 ml of toluene and dried at 60° C. in a circulating air dryer.

Yield: 154.6 g (96%)
Colourless crystals

| | | | | |
|---|---|---|---|---|
| C₃₃H₂₉N₄O₂Na × 0.5H₂O | calc.: | C 72.51 | H 5.72 | N 10.25 |
| | found: | C 72.57 | H 5.69 | N 10.21 |

### Example 6

### Preparation of Crystalline Telmisartan Sodium Salt from Telmisartan Hydrochloride

Preparation of Telmisartan-Hydrochloride:

411 g of tert.-butyl-4′-[[2-n-propyl-4-methyl-6-(1-methylbenzimidazol-2-yl)-benzimidazol-1-yl]-methyl]-biphenyl-2-carboxylate were suspended in 822 ml glacial acetic acid and combined with 213 g concentrated aqueous hydrochloric acid (37%). The mixture was refluxed. About 640 ml of the solvent were distilled off. The residue remaining was slowly combined with about 620 ml of water at 50-60° C. This mixture was combined with 20 g activated charcoal (e.g. Norit SX 2 Ultra). The mixture obtained was stirred for about 10 minutes at constant temperature. After filtering, the residue was washed 3 times with 25 ml of glacial acetic acid and about 620 ml of water. The filtrate obtained was again heated to about 50-60° C. and combined with about 2 litres of water. After about 12 hours' stirring at about 23° C. the crystals formed were suction filtered and washed twice with about 500 ml of water and once with about 900 ml acetone and then dried at about 60° C.

Yield: 367 g (92.5%)

Colourless crystals

melting point: 278° C.

Preparation of Crystalline Telmisartan Sodium Salt from Telmisartan-Hydrochloride:

55.1 g telmisartan-hydrochloride were taken up in 110.2 ml of toluene, 5.5 ml of water and 55.1 ml isopropanol. This mixture was combined with 36.9 g sodium methoxide (30% in methanol) and 2.75 g activated charcoal (e.g. Norit SX 2 Ultra). Then, the mixture was heated to about 75° C. About 50 ml of the solvent mixture were distilled off at constant temperature within about 30 minutes. The suspension obtained was filtered. The residue was washed with about 20 ml of toluene. The filtrate was combined with about 5 ml of water and about 150 ml of toluene. The mixture obtained was refluxed. About 150 ml of solvent mixture were distilled off azeotropically (up to 102° C.). The mixture was left to crystallise for 1 hour at 100° C. The crystals were suction filtered, washed with about 50 ml of toluene and dried at about 60° C.

Yield: 53.6 g (99%)
Colourless crystals

| | | | | |
|---|---|---|---|---|
| C₃₃H₂₉N₄O₂Na.0.5H₂O | calc.: | C 72.51 | H 5.72 | N 10.25 |
| | found: | C 72.44 | H 5.68 | N 10.20 |

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims. Various patent applications and publications are cited herein, the disclosures of which are incorporated by reference in their entireties.

What is claimed is:

1. A method for the prevention or treatment of cardiovascular, cardiopulmonary or renal disease in a mammal comprising administering a pharmaceutical composition comprising telmisartan or one of the salts thereof and atorvastatin.

2. A method for the prevention or treatment of hypertension combined with hyperlipidaemia or atherosclerosis in a mammal comprising administering a pharmaceutical composition comprising telmisartan or one of the salts thereof and atorvastatin.

3. A method for the treatment of asthma, bronchitis or interstitial lung disease in a mammal comprising administering a pharmaceutical composition comprising telmisartan or one of the salts thereof and atorvastatin.

4. A method for the prevention or treatment of diabetes mellitus or prediabetes in a mammal comprising administering a pharmaceutical composition comprising telmisartan or one of the salts thereof and atorvastatin.

5. A method for the treatment of metabolic syndrome and insulin resistance in a mammal with normal blood pressure comprising administering a pharmaceutical composition comprising telmisartan or one of the salts thereof and atorvastatin.

6. A method for the prevention or treatment of hypertensive insulin resistance in a mammal comprising administering a pharmaceutical composition comprising telmisartan or one of the salts thereof and atorvastatin.

7. The method according to claim 1, wherein the mammal is a human.

8. The method according to claim 1, wherein a fasting blood sugar level of the mammal exceeds 110 mg of glucose per dl of plasma.

9. The method according to claim 1, wherein a blood level for triglyceride of the mammal exceeds 150 mg/dl.

10. The method according to claim 7, wherein a blood level for high-density lipoprotein in the human is less than 40 mg per dl of plasma in a female human and less than 50 mg per dl of plasma in a male human.

11. The method according to claim 4, wherein the systolic blood pressure of the mammal exceeds a value of 130 mm Hg and the diastolic blood pressure of the mammal exceeds a value of 80 mm Hg.

12. The method according to claim 10, wherein the atorvastatin or a polymorph or salt thereof is administered orally in a daily dose of about 0.018 mg/kg body weight to 6.43 mg/kg body weight and the telmisartan or a polymorph or salt thereof is administered orally in a daily dose of about 0.143 mg/kg to 7.143 mg/kg body weight.

13. The method according to claim 10, wherein the atorvastatin or a polymorph or salt thereof is administered parenterally in a daily dose of about 0.286 mg/kg body weight and the telmisartan or a polymorph or salt thereof is administered parenterally in a daily dose of about 0.286 mg/kg body weight.

14. A pharmaceutical composition, comprising telmisartan and atorvastatin, optionally with one or more excipients for the prevention or treatment of cardiovascular, cardiopulmonary or renal diseases.

15. The pharmaceutical composition according to claim 14, wherein the formulation of the pharmaceutical composition comprises 20-200 mg telmisartan and 2.5-40 mg atorvastatin.

16. The pharmaceutical composition according to claim 15, wherein a ratio of atorvastatin to telmisartan or a polymorph or salt thereof is 1:2 to 1:8 (wt/wt).

17. The pharmaceutical composition according to claim 14, further comprising a diuretic.

18. The pharmaceutical composition according to claim 17, wherein the diuretic consists of 10-50 mg of hydrochlorothiazide or 10-50 mg of chlorthalidone.
